(19) United States
(12) Patent Application Publication
(10) Pub. No.: US 2009/0142398 A1
(43) Pub. Date: Jun. 4, 2009

(54) NOVEL PHARMACEUTICAL COMPOSITIONS COMPRISING A DISINTEGRATION MATRIX

(75) Inventors: Mathew Philip, St-Laurent (CA); Naresh Talwar, Kirkland (CA); Rasik Patel, St-Laurent (CA); Vinayak Pathak, Montreal (CA)

Correspondence Address: STIKEMAN ELLIOTT LLP, 1600-50 O''CONNOR STREET, OTTAWA, ON, KIP 6L2, CA

(73) Assignee: Pharmascience Inc., Montreal (CA)

(21) Appl. No.: 12/273,937

(22) Filed: Nov. 19, 2008

Related U.S. Application Data
(60) Provisional application No. 61/003,977, filed on Nov. 21, 2007.

(51) Int. Cl. A61K 9/10; A61K 31/4184

(52) U.S. Cl. 424/484; 514/394; 424/486

(57) ABSTRACT

The present invention relates to a stable pharmaceutical composition comprising a pharmaceutically active substance having poor water solubility dispersed in a pharmaceutically acceptable disintegration matrix, said disintegration matrix comprising at least one pharmaceutically acceptable disintegrant, a pharmaceutically acceptable basic agent provided in a molar ratio of basic agent to active substance of 1:1 to 10:1, a water-insoluble pharmaceutically acceptable diluent, optionally, if desired or necessary at least one pharmaceutically acceptable excipients and/or pharmaceutically acceptable adjuvants, and optionally a pharmaceutically acceptable surfactant or emulsifier. The present invention also provides a process to make such.

### CROSS-REFERENCE TO RELATED APPLICATIONS

This application claims the benefit of U.S. Provisional Application Ser. No. 61/003,977 filed Nov. 21, 2007.

### FIELD OF THE INVENTION

The present invention relates to a novel pharmaceutical composition comprising an angiotensin II receptor antagonist, as well as formulations made from said pharmaceutical composition. The present invention also provides methods for producing said compositions.

### BACKGROUND OF THE INVENTION

Inhibition of the rennin angiotensin system is a well-proven approach to the treatment of arterial hypertension. It can be achieved by inhibiting the angiotensin-converting enzyme (ACE) that converts angiotensin I into its active form angiotensin II (AGII), or by blockade of angiotensin II (type AT1) receptors. To achieve this result, angiotensin II receptor blockers (ARBs) or antagonists, belonging to the therapeutic class of antihypertensive agents (BUMC Proceedings 2003; 16: 123-126), typically bind to the angiotensin II type 1 (AT1) receptors with high affinity, causing inhibition of the action of angiotensin II on vascular smooth muscle, ultimately leading to a reduction in arterial blood pressure.

Such ARBs include: candesartan, eprosartan, irbesartan, losartan, olmesartan, telmisartan and valsartan.

Belonging to this therapeutic category of ARBs are certain pharmaceutical active substances, such as candesartan, eprosartan, irbesartan, losartan, olmesartan, telmisartan and valsartan (Merck Index 14th edition). It is known that telmisartan, for example, falls under the general class of chemical compounds known as benzimidazoles, which have pharmaceutically useful properties, especially as an angiotensin antagonist. Such compounds are, for example, disclosed in Canadian patent no. 2,060,624 (issued to Dr. Karl Thomae GmbH on Dec. 21, 1999).

Telmisartan, known under the trade names Micardis®, Kinzalmono®, and Pritnor®, is chemically designated as either 4′-[(1,4′-dimethyl-2′-propyl[2,6′-bi-1H-benzimidazol]-1yl)methyl][1,1′biphenyl]-2-carboxylicacid or 4′-[[4-methyl-6-(1-methyl-2-benzimidazolyl)-2-propyl-1-benzimidazoly]methyl]-2-biphenylcarbo-xylic acid. It has an empirical formula of C₃₃H₃₀N₄O₂ as well as a molecular weight of 514.62 (C 77.02%, H 5.88%, N 0.89%, O 6.22%).

Telmisartan can be represented by the following chemical structure:

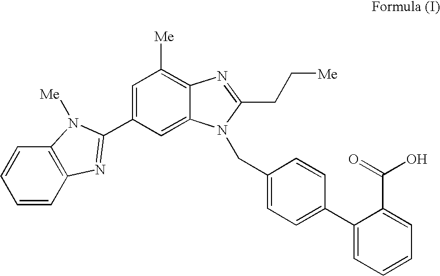

The preparation of telmisartan was published in European patent no. EP 0 502 314 B1 to Dr. Karl Thomae GmbH on Sep. 9, 1992, as well in the Journal of Medicinal Chemistry 36,4040 (1993) by U. J. Ries et al. Corresponding to this European patent is Canadian patent no. 2,060,624, referred to above, and U.S. Pat. No. 5,591,762, issued on Jan. 9, 1997. Both of these patents disclose telmisartan and a use thereof for the treatment of various diseases. It is worth mentioning that many U.S. patent equivalents have derived from European patent no. EP 0 502 314 B1, including U.S. Pat. Nos. 5,594,003; 5,602,127 and 5,614,519, all in the name of Dr. Karl Thomae GmbH.

It is known that the main physicochemical properties of telmisartan are low solubility, high lipophilicity and polymorphism, as well, when in combination with soluble excipients and basic agents, hygroscopicity; thus rendering the formulation susceptible to absorbing water from the environment.

Canadian patent application no. 2,499,878 (filed Sep. 18, 2003 by Boehringer Ingelheim International GmbH), for example, describes a composition which allows telmisartan to be released with sufficient solubility for gastrointestinal absorption in a slightly acidic and neutral pH region. The composition disclosed comprises from 3 to 50 wt. % of telmisartan, telmisartan being dispersed in a dissolving matrix comprising:
(a) a basic agent in a molar ratio of basic agent: telmisartan ranging from 1:1 to 10:1;
(b) a surfactant or emulsifier in an amount of about 1 to 20 wt. % of the final composition;
(c) 25 to 70% wt. of a water-soluble diluent, and
(d) optionally 0 to 20% wt. of further excipients and/or adjuvants, the sum of all components equalling 100% by weight of the final composition.

A problem associated with the composition taught in Canadian patent application no. 2,499,878 is that it remains highly hygroscopic; thus sensitive to moisture and requires specialized packaging to prevent moisture absorption on shelf life. Moreover, the end product (or composition), in a dissolving matrix, is made by a process which uses either spray drying or a fluid bed granulation techniques, which necessarily involve the step of dissolving telmisartan in a sodium hydroxide or meglumine solution. By using either of these techniques, the manufacturer can use dilute concentrations of telmisartan solutions, containing sodium hydroxide or meglumine, so that it can be appropriately granulated. Furthermore, by using a dissolving matrix it is difficult to manufacture telmisartan compositions by using conventional granulation techniques, as many issues, for example, granulation end point control, granule properties, etc., have to be addressed. It would thus be advantageous to have a composition which is relatively insensitive (poorly hygroscopic) to moisture, and that can be manufactured by less expensive process without affecting the quality of the product.

There is thus a need to have a composition and/or formulation containing angiotensin II receptor antagonists, for example telmisartan, which can be prepared using relatively uncomplicated and inexpensive process techniques and have desirable prerequisites for pharmaceutical use, i.e. long-lasting stability of the formulation under different environmental conditions and sufficient solubility of the active substance.

It has surprisingly been found that the use of a disintegration matrix is beneficial, as opposed to the use of a dissolving matrix. Indeed, the disintegration matrix according to the present invention significantly reduces the hygroscopicity of the finished formulation to a great extent without adversely affecting the quality of the product, for example the dissolution rate of the formulation.

It has also been surprisingly found that by using a disintegration matrix in the composition of the present invention, the problems associated with various manufacturing processes of the prior art have been overcome. Indeed, by using a disintegration matrix in the composition of the present invention, a manufacturer is enabled to produce, for example, a telmisartan composition with conventional processing methods, such as low shear granulation.

It has also surprisingly been found that when a disintegration matrix is used in the composition and/or formulation of the present invention, it provides the additional advantages of not requiring the end product (i.e. a dosage form, such as tablets, capsules, etc.) to be packaged in specialized packaging and does not require a reference to special precautions or directives on how to handle the packaged product.

### SUMMARY OF THE INVENTION

Thus, in accordance with a general aspect, the present invention provides a pharmaceutical composition comprising a pharmaceutically active substance dispersed in a disintegration matrix.

Stated otherwise, the present invention relates to a pharmaceutical composition comprising a pharmaceutically active component and a pharmaceutically acceptable excipient component, characterized in that the excipient component comprises a disintegration matrix, the pharmaceutically active component being dispersed in said disintegration matrix.

One aspect of the present invention is to provide a pharmaceutical composition comprising a pharmaceutically active substance dispersed in a pharmaceutically acceptable disintegration matrix, said disintegration matrix comprising:
at least one pharmaceutically acceptable disintegrant;
a pharmaceutically acceptable basic agent provided in a molar ratio of basic agent to active substance is in ratio of 1:1 to 10:1;
a water-insoluble pharmaceutically acceptable diluent;
optionally, if desired or necessary at least one (e.g. other) member of the group consisting of pharmaceutically acceptable excipients and/or pharmaceutically acceptable adjuvants, and
optionally a pharmaceutically acceptable surfactant or emulsifier.

The disintegration matrix of the present invention may contain a plurality of components, such as for example, disintegrants, basic agents, water insoluble diluents and optionally if desired or as necessary, at least one (e.g. other) pharmaceutically acceptable excipients and/or adjuvant. The disintegration matrix may further comprise a surfactant and/or an emulsifier.

The present invention in particular relates to a pharmaceutical composition wherein the active substance may be an angiotensin II receptor antagonist. Thus the pharmaceutical composition may comprise a pharmaceutically active substance which is candesartan, eprosartan, irbesartan, losartan, olmesartan, telmisartan, valsartan or a mixture thereof; in preferably the pharmaceutically active substance may be telmisartan.

In one aspect of the application, the present invention relates to a pharmaceutical composition wherein the disintegrant is croscarmellose sodium. The basic agent in the disclosed pharmaceutical composition may be selected from alkali metal hydroxides, NaHCO₃, KHCO₃, Na₂CO₃, 15 Na₂HPO₄, OHPOO, tromethamine, triethanolamine, MgO, MgCO₃ and basic aminoacids such as meglumine and arginine. Preferably, the basic agent is an alkali metal hydroxide, more preferably, the basic agent is selected from the group consisting of sodium hydroxide or potassium hydroxide.

In a further aspect of the present invention, the water-insoluble diluent of the pharmaceutical composition may be selected from microcrystalline cellulose, di- or tri-basic calcium phosphate, meglumine oxide, crystalline cellulose, powdered cellulose, anhydrous silicic acid, calcium carbonate, calcium sulphate, magnesium silicate, magnesium trisilicate, magnesium aluminium metasilicate (Neusilin™), kaolin, starch and starch derivatives, magnesium carbonate, magnesium oxide and co-processed insoluble excipients. The present invention relates in particular to a pharmaceutical composition wherein the co-processed insoluble excipient may be silicified microcrystalline cellulose.

In another aspect, the present invention relates to a pharmaceutical composition wherein the (e.g. other) pharmaceutical excipients and/or adjuvants may be selected from binders, carriers, lubricants, flow agents, adsorbants, crystallization retarders, solubilizers, antiadherents, surfactants, emulsifiers, pH modifiers and colouring agents. The present invention in particular relates to a pharmaceutical composition wherein the flow agent may be colloidal silicon dioxide.

More particularly the present invention provides a pharmaceutical composition comprising 5.0% to 50% wt of telmisartan dispersed in a disintegration matrix, said disintegration matrix comprising:
at least one disintegrant in an amount 0.5%-20% wt (e.g. 2%-20% wt);
a basic agent provided in a molar ratio of basic agent to active substance in the range of 1:1 to 10:1;
a water-insoluble diluent in an amount 15%-75% wt;
at least one member of the group consisting of pharmaceutically acceptable excipients and/or pharmaceutically acceptable adjuvants, in an amount 0-25% wt; and
optionally a surfactant or emulsifier 0.5%-10% wt (e.g. 2%-10% wt), the sum of all components equalling 100%.

In accordance with a further aspect, the present invention provides a formulation comprising the aforementioned composition in the form of a tablet or capsule.

In accordance with a further aspect the present invention provides a process for producing said compositions and formulations mentioned hereinabove.

Other objects and advantages of the present invention will be apparent upon reading the following non-restrictive description of several preferred embodiments, made with reference to the accompanying figures, drawings and/or examples.

### BRIEF DESCRIPTION OF THE DRAWINGS

The embodiments of the present invention are described below with reference to the accompanying drawings in which:

FIG. 1 is a diagram illustrating the dissolution profile of telmisartan in a phosphate buffer at pH 7.5

### DETAILED DESCRIPTION OF THE PRESENT INVENTION

It is to be understood herein that the expression “dissolving matrix” and/or “dissolution matrix” as used in the present application generally refers to a matrix containing water soluble pharmaceutically acceptable excipients, and releases an active ingredient by dissolution of the matrix. Dissolving matrices of the prior art may or may not contain a disintegrant.

It is to be understood that the expression “disintegration matrix” as used in the present application generally refers to a matrix which is generally made up of excipients, insoluble or other, and is capable of disintegrating into finer particles by the effect of suitable disintegrants in the matrix which may be done by swelling, wicking deformation or chemical reaction. Suitable disintegrants may be, for example, those defined in Remington: The Science and Practice of Pharmacy (20th edition, 2000), though other disintegrants, as referred to herein, can be selected. Indeed, it can be noted, from the following detailed description and/or examples, that certain excipients in the formulation do not have to be insoluble (i.e. sodium hydroxide, meglumine, povidone), but that some of them can also be used as diluents, disintegrants, flow aids and lubricants, which may be by their nature insoluble or not. Certain excipients, by their nature, are multi-functional.

The main difference between a dissolving matrix and a disintegration matrix consists in the choice of pharmaceutically acceptable excipients. For example, in the case of a dissolving matrix, soluble excipients will be used. In the case of a disintegrating matrix, insoluble excipients should be used. It is worth mentioning that both of these matrices may contain other common components, such as basic agents (i.e. sodium hydroxide and meglumine) or any other agents, such as lubricants (i.e. magnesium stearate). The appropriate choice of disintegration matrix is important. Criterion used to choose such disintegration matrices include the ease of manufacturing by conventional methods (i.e. low shear granulation), cost (i.e. cost effectiveness) and processing speed (i.e. low processing time). Formulations derived from dissolving matrices often involve high processing costs and are more time consuming, such being due to the use of spray drying or fluid bed granulation techniques. Indeed, as opposed to conventional dissolution matrices, disintegration matrices allow for the rapid disintegration of the matrix into finer granules which thereby enables a good dissolution rate of telmisartan in physiological media. For further details, reference can be made to Table 1 below.

In accordance with the present invention the word “excipient” is to be understood as referring to any component of a pharmaceutical composition or finished drug dosage form other than the mentioned therapeutic ingredient or ingredients. Thus, for example an excipient may be (i) any inert material that is combined with an active drug in order to produce a drug dosage form; (ii) an inactive ingredient added to a drug (i.e. in a pill form) to dilute it or to give it form or consistency; and/or (iii) the filler portion of the final drug product, often an inert substance so as not to interact with the API. It may also comprise an adjuvant.

In accordance with the present invention the word “adjuvant” is to be understood as being a pharmacological agent added to a drug formulation to enhance its effect. Thus, an adjuvant may for example be:
(i) a substance that, when added to a medicinal agent or a pharmaceutical active substance, speeds or improves its action (auxiliary remedy);
(ii) any component which improves the effect of a drug or immunological agent; and/or
(iii) an ingredient (as in a prescription or solution) that facilitates or modifies the action of the principal ingredient.

It is also to be understood herein that any reference to percentage or percentages is mentioned on a weight per weight basis unless the contrary is indicated or is mandated by the context.

In accordance with the present invention the pharmaceutically active substance of the present invention may be an angiotensin II receptor antagonist. In accordance with the present invention the angiotensin II receptor antagonist may belong to any of the chemical compounds belonging to the group of benzimidazoles or derivatives thereof. Thus, for example the active substance may be selected from the group consisting of candesartan, eprosartan, irbesartan, losartan, olmesartan, telmisartan and valsartan (Merck Index 14th edition) as well as mixtures thereof. It is of course to be understood herein that any salt, ester, analog, and pro-drugs of an active substance may be exploited in accordance with the present invention. In this vein, telmisartan sodium, candesartan cilexitil, losartan potassium, etc. may be suitable choices of pharmaceutically active substance.

According to the present invention, the pharmaceutically active substance, for example telmisartan, may be in a ratio of 5.0 to 50 wt. % of the total composition.

A pro-drug is to be understood herein as being a pharmacological substance (e.g. drug) which is administered in an inactive (or significantly less active) form; once administered, the pro-drug is metabolised in the body (in vivo) into the active compound. Stated in another way, a pro-drug is an inactive precursor of a drug, converted into an active form in the body by normal metabolic processes.

As used herein, the term “pharmaceutically acceptable salt” refers to salts that are physiologically tolerated by a user.

In accordance with the present invention, disintegrants which are known in the art may be used and include, and are not limited to, hydroxypropyl starch, alginic acid, calcium alginate, carboxymethylcellulose calcium, carboxymethylcellulose sodium, cellulose, chitosan, colloidal silicon dioxide, croscarmellose sodium, erospovidone, docusate sodium, guar gum, hydroxypropyl cellulose, low-substituted hydroxypropyl cellulose, magnesium aluminium silicate, methylcellulose, microcrystalline cellulose, polacrilin potassium, povidone, sodium alginate, sodium starch glycolate, starch and pregelatinized starch. Preferably, according to the present invention, croscarmellose sodium may be used in the disintegration matrix of the pharmaceutical composition as it exhibits superior disintegration properties through wicking as opposed to other disintegrants which undergo swelling. According to the present invention, the at least one disintegrant may be in an amount of 2.0 to 20 wt. % of the total composition.

In accordance with the present invention, the basic agent may be selected from the group consisting of: alkali metal hydroxides, NaHCO₃, KHCO₃, Na₂CO₃, Na₂HPO₄, K₂HPO₄, tromethamine or a salt thereof, triethanolamine, MgO, MgCO₃ and basic amino acids, as well as mixtures thereof. For example, the alkali metal hydroxide may be selected from sodium hydroxide or potassium hydroxide or mixtures thereof. In cases wherein the basic agent is an amino-acid, the basic agent may be selected from the group of meglumine and arginine. In a particular aspect, the basic agent may be provided in a molar ratio of basic agent to active substance in a ratio of from 1:1 to 10:1.

In accordance with the present invention, the water insoluble diluent of the present invention may be selected from a variety of chemical compounds including microcrystalline cellulose, di- or tri-basic calcium phosphate, crystalline cellulose, powdered cellulose, anhydrous silicic acid, calcium carbonate, calcium sulphate, magnesium silicate, magnesium trisilicate, magnesium aluminium metasilicate (Neusilin™), kaolin, starch and starch derivatives, magnesium carbonate, magnesium oxide and co-processed insoluble excipients.

Co-processed excipients are excipients which are obtained by co-processing technique. Co-processing is a novel concept wherein two or more excipients are intermingled at a sub-particle level. The main objective of co-processing is to provide a synergy of functionality improvements to the excipient, as well as masking the undesirable properties of individual excipients. Co-processed excipients may be manufactured by spray drying or flash drying process. As co-processed excipients are multifunctional, they can be used at different levels in the formulation.

Examples of co-processed excipients, used in pharmaceutical industry, include, for example: Ludipress® (mixture of lactose and 3.2% Povidone K₃₀), Cellactose® (mixture of lactose and cellulose), Prosolv® (mixture of microcrystalline cellulose and silicon dioxide). The article entitled “Coprocessed excipients for Solid dosage forms” by Arvind. K. and Bhansal et al. (Pharmaceutical Technology, 2004) describes co-processed excipients. In the case that a co-processed insoluble excipient is selected from the list of water insoluble diluents, silicified microcrystalline cellulose (Prosolv®) may be used by way of example. However, it is worth mentioning that any other insoluble diluent which can increase the bulk of the formulation may be used.

According to the present invention, the water insoluble diluent may be present in an amount of 15 to 75 wt. % of the total composition.

In accordance with the present invention, the at least one pharmaceutically acceptable excipients and/or adjuvants may be selected from the group consisting of binders, carriers, lubricants, flow agents, adsorbants, crystallization retarders, disintegrants, solubilizers, anti-adherents, surfactants, pH modifiers and coloring agents. For an overview of excipients used in the pharmaceutical industry, reference can be made to the Handbook of Pharmaceutical Excipients (5th edition) by Raymond C. Rowe, Paul J. Sheskey and Sian C. Owen.

Preferably, according to the present invention, at least one member of the group consisting of pharmaceutically acceptable excipients and pharmaceutically acceptable adjuvants is in an amount ranging from 0 to 25 wt. % of the total composition.

Indeed, in the case that a flow agent is desired to be used, colloidal silicon dioxide may be used. Similarly, if an adsorbant is desired to be used, magnesium aluminium metasilicate may be used.

It is also worth mentioning that if any surfactant or emulsifier is used in the composition of the present invention, such may be in an amount of 2 to 10 wt. % of the total composition. Examples of emulsifiers are MYVACET™ (distilled acetylated mono-glyceride emulsifers); ARLACEL™ (mainly sorbitan esters); TWEEN™ (polyoxyethylene sorbitan esters); CENTROPHASE™ (fluid lecithins); CREMOPHOR™ (polyoxyl castor oil derivatives; or macrogol ethers; or macrogol esters); LABRAFAC™ (caprylic/capric triglyceride); LABRAFIL™ (polyoxyethylated glycolysed glycerides); and LABRASOL™ (mixture of mono-, di- and triglycerides and mono- and di-fatty esters of polyethylene glycol). The predominant fatty acids are C₈-C₁₀ caprylic/capric acids; MYVEROL™; and TAGAT™ (polyethyleneglycol hydrogenated castor oil; or polyethyleneglycol glyceryl esters); lecithin; and proteins such as casein.

Suitable examples for surfactants include, and are not limited to, sodium lauryl sulphate, polyoxyethylene sorbitan fatty acid esters (TWEEN™ series), Myrj™ series, solutol HS, polyoxyethylene alkyl ethers (for example the Brij™ series), polyoxyethylene castor oil derivatives, polyoxyethylene stearates, sorbitan esters (sorbitan fatty acid esters), poloxamers, sucrose fatty acid ester, vitamin E TPGS, and polyethylene glycol fatty acid esters.

It is worth mentioning that since most pharmaceutical excipients are multifunctional, some of the excipients may be repeated in different categories mentioned both hereinabove and below.

Once the pharmaceutical according to the present invention has been prepared, it can be used to make a solid oral formulation, usually in the form of a capsule or tablet. This solid oral form can optionally be film coated with a film forming polymer. The film forming polymer may be selected from hydroxypropylmethylcellulose, ethylcellulose, polyvinyl alcohol, hydroxypropylcellulose, acrylic polymers (Eudragit®), hydroxyethylcellulose, polyvinyl pyrrolidone (Povidone), vinyl pyrrolidone-vinylacetate copolymer (copovidone) as well as mixtures thereof.

### Manufacturing Process

In accordance with the present invention a composition may be prepared by a general granulation process characterized by the following steps:
(A) preparing a dry mixture containing at least one water insoluble diluent (preferably between 15-75%), an adsorbant (preferably between 10%-25.0%), a flow aid (preferably between 1.0%-5.0%), and a disintegrant (1.0-10.0%);
(B) preparing a granulation (or drug-binder) solution, said solution obtained by combining a first mixture containing purified water, at least one basic agent (1:1 to 1:10 molar ratio of basic agent to telmisartan) and a pharmaceutically active substance (preferably between 5-50%), to a second mixture containing a binder (preferably between 1-10%) dissolved in a alcohol;
(C) screening the dry mixture obtained in step (A) through a screen and then charging it into a low shear equipment;
(D) granulating the dry mixture of steps (A) and (C) with granulation solution of step (B) so as to form granules;
(E) drying the so formed granules of step (D) in a fluid bed drier or a tray drier;
(F) co-milling the dried granules of step (E) through a co-mill;
(G) preparing a mixture containing a basic agent (preferably between 1-10%), a water insoluble diluent (preferably between 10%-30%) and a disintegrant (preferably between 1.0-10%), all of which will be screened through a sieve;
(H) mixing the mixture of step (G) with the dried granules of step (F) to form a mixture to which a lubricant (preferably between 0.25%-2.0%) is added thereon;
(I) compressing said mixture of step (H) into a solid dosage form; and
(J) coating the compressed tablets with Opadry® (preferably between 1.0-3.0% wt gain) (Colorcon, U.S.A.).

### EXAMPLES

The following examples are illustrative of the wide range of applicability of the present invention and are not intended to limit its scope. Modifications and variations can be made therein without departing from the spirit and scope of the invention.

Formulation A

| | | | |
|---|---|---|---|
| Serial | | % | Qty |
| # | Ingredient | concentration | (gm) |

Dry Mixing

| | | | |
|---|---|---|---|
| 1 | Silicified Microcrystalline cellulose | 25.0 | 125.0 |
| | 90 (Prosolv ®) | | |
| 2 | Dibasic calcium Phosphate | 30.0 | 150.0 |
| 3 | Colloidal silicon dioxide | 1.0 | 5.0 |
| 4 | Croscarmellose sodium | 4.0 | 20.0 |

Granulation

| | | | |
|---|---|---|---|
| 5 | Telmisartan | 16.66 | 83.3 |
| 6 | Sodium Hydroxide | 1.38 | 6.9 |
| 7 | Purified water | — | 110.0 |
| 8 | Povidone (K-30) | 3.0 | 15.0 |
| 9 | Isopropyl alcohol | — | 35.0 |

Lubrication

| | | | |
|---|---|---|---|
| 10 | Microcrystalline cellulose (Avicel Ph | 14.04 | 70.2 |
| | 102) | | |
| 11 | Croscarmellose sodium | 4.0 | 20.0 |
| 12 | Magnesium stearate | 1.0 | 5.0 |
| | Total | 100.0 | 500.0 |

Formulation A: Manufacturing procedure

Formulation A, a preferred embodiment of the present invention, was made by following the steps set out below:
1) Ingredients 1, 2, 3 & 4 are screened through a suitably sized screen (i.e. 850μ) and charged into low shear equipment (Planetary mixer) and mixed for a predetermined amount of time to obtain a homogenous blend. The aforementioned ingredients may be mixed for 5 minutes; though this amount of time may vary based on batch size and the equipment used.
2) A solution of telmisartan (83.3 g) and sodium hydroxide (6.9 g) is made in purified water (110.0 g) under continuous stirring at, for example, room temperature (i.e. 25° C.).
3) A solution of Povidone K-30 (15.0 g) is made in isopropyl alcohol (35.0 g) at, for example, room temperature (i.e. 25° C.). Both the solutions of steps 2 & 3 are combined together and the resulting solution is granulated over the blend of step 1.
4) The granulated blend was dried in a tray dryer (Shell Lab) at an inlet temperature of 50° C. till a loss of drying (“LOD”) value of 3.5-4.5% w/w is obtained.
5) The dried granules are milled (Co-mill) through a, for example, 0.039 inch screen to obtain a uniform sized granules.

It is worth mentioning that milling is a process by which we are screening the granules through a screen of any size to obtain granules which are uniform in size. If milling is done after granulation, this would help in uniform drying of granules. This, of course, depends on the formulator to decide whether the process requires milling or not. In the present case, after granulation, the granules were not milled. In fact, the granules were directly dried on the tray drier. But if drying was done on a fluid bed, the process involves milling the granules at a semidried condition. The semidried condition is where a LOD value between 7-8% w/w is obtained. All the examples provided in the present patent application are with the tray dryer.
6) The dried and screened granules are then blended in a suitable blender with ingredients 10 and 11, which was pre-screened through, for example, a 850μ sized screen and further lubricated with ingredient 12, which was pre-screened through, for example, a 425μ screen.
7) The resulting blend is compressed into tablets on a Colton rotary machine using capsule shaped punches.

Formulation B & C
Lot 500 gm

| | | | |
|---|---|---|---|
| | | % | |
| Serial # | Ingredient | concentration | Qty (gm) |

Dry Mixing

| | | | |
|---|---|---|---|
| 1 | Silicified Microcrystalline cellulose | 25.0 | 175.0 |
| | 90 (Prosolv ®) | | |
| 2 | Dibasic calcium Phosphate | 15.0 | 75.0 |
| 3 | Colloidal silicon dioxide | 1.0 | 5.0 |
| 4 | Croscarmellose sodium | 4.0 | 20.0 |

Granulation

| | | | |
|---|---|---|---|
| 5 | Telmisartan | 16.66 | 83.3 |
| 6 | Sodium Hydroxide | 1.38 | 6.9 |
| 7 | Purified water | — | 110.0 |
| 8 | Povidone (K-30) | 3.0 | 15.0 |
| 9 | Isopropyl alcohol | — | 35.0 |

Lubrication (Qty for 250 g) Formula B

| | | | |
|---|---|---|---|
| 10 | Co-milled granules (from Step 5 of | 76.04 | 190.1 |
| | manufacturing procedure) | | |
| 11 | Magnesium Oxide | 5.0 | 12.5 |
| 12 | Microcrystalline cellulose (Avicel | 14.0 | 35.0 |
| | Ph 102) | | |
| 13 | Croscarmellose sodium | 4.0 | 10.0 |
| 14 | Magnesium stearate | 1.0 | 2.5 |
| | Total | 100.0 | 250.0 |

Lubrication (Qty for 250 g) Formula C

| | | | |
|---|---|---|---|
| 15 | Co-milled granules (from Step 5 of | 76.04 | 190.1 |
| | manufacturing procedure) | | |
| 16 | Magnesium Oxide | 5.0 | 12.5 |
| 17 | Microcrystalline cellulose (Avicel | 14.0 | 35.0 |
| | Ph 102) | | |
| 18 | Croscarmellose sodium | 4.0 | 10.0 |
| 19 | Magnesium stearate | 1.0 | 2.5 |
| | Total | 100.0 | 250.0 |

### Formulation B & C: Manufacturing Procedure Till Drying Stage

1) Ingredients 1, 2, 3 & 4 are screened through a suitably sized screen (i.e. 850μ) and charged into low shear equipment (Planetary mixer) and mixed for approximately minutes to obtain a homogenous blend.

2) A solution of Telmisartan (83.3 g) and sodium hydroxide (6.9 g) is made in purified water (110.0 g) under continuous stirring at, for example, room temperature (i.e. 25° C.).

3) A solution of Povidone K-30 (15.0 g) is made in isopropyl alcohol (35.0 g) at, for example, room temperature (i.e. 25° C.). Both the solutions of steps 2 & 3 are combined together and the resulting solution is granulated over the blend of step 1.

4) The granulated blend was dried in a tray dryer (Shell Lab) at an inlet temperature of 50° C. till a loss of drying (“LOD”) value of 2.5-3.5% w/w is obtained.

5) The dried granules are milled (Co-mill) through a, for example, 0.039 inch screen to obtain a uniform sized granules.

6) The co-milled granules were divided into two sub lots (i.e. sub lot A and sub lot B).

### Sub Lot A (250.0 g) Corresponding to Formulation B

7a) The dried and screened granules resulting from step 6 are then blended with ingredients 10, 11 and 12, which were pre-screened through, for example, a 850μ sized screen and further lubricated with ingredient 13 which was pre-screened through, for example, a 425μ screen.

8a) The resulting blend is compressed into tablets on a Colton rotary machine using capsule shaped punches.

### Sub Lot B (250.0 g) Corresponding to Formulation C

7b) The dried and screened granules (one sub lot) resulting from step 6 are then blended with ingredients 16, 17 and 18, which were pre-screened through, for example, a 850μ sized screen and further lubricated with ingredient 19 which was pre-screened through, for example, a 425μ screen.

8b) The resulting blend is compressed into tablets on a Colton rotary machine using capsule shaped punches.

Formulation D

| | | | |
|---|---|---|---|
| | | % | |
| Serial # | Ingredient | concentration | Qty (gm) |

Dry Mixing

| | | | |
|---|---|---|---|
| 1 | Silicified Microcrystalline cellulose | 35.0 | 87.5 |
| | 90 (Prosolv ®) | | |
| 2 | Tri Basic calcium Phosphate | 15.0 | 37.5 |
| 3 | Colloidal silicon dioxide | 1.0 | 2.5 |
| 4 | Croscarmellose sodium | 4.0 | 10.0 |

Granulation

| | | | |
|---|---|---|---|
| 5 | Telmisartan | 16.66 | 41.65 |
| 6 | Sodium Hydroxide | 1.38 | 3.45 |
| 7 | Meglumine | 5.0 | 12.5 |
| 8 | Purified water | — | 73.0 |
| 9 | Povidone (K-30) | 3.0 | 7.5 |
| 10 | Isopropyl alcohol | — | 20.0 |

Lubrication

| | | | |
|---|---|---|---|
| 11 | Microcrystalline cellulose (avicel | 14.0 | 35.0 |
| | Ph 102) | | |
| 12 | Croscarmellose sodium | 4.0 | 10.0 |
| 13 | Magnesium stearate | 1.0 | 2.5 |
| | Total | 100.0 | 250.0 |

### Formula D: Manufacturing Procedure

1) Ingredients 1, 2, 3 & 4 are screened through a suitably sized screen (i.e. 850˜) and charged into low shear equipment (Planetary mixer) and mixed for approximately 5 minutes to obtain a homogenous blend.

2) A solution of Telmisartan (41.65 g), meglumine (12.5 g) and sodium hydroxide (3.45 g) is made in purified water (73.0 g) under continuous stirring at, for example, room temperature (i.e. 25° C.).

3) A solution of Povidone K-30 (7.5 g) is made in isopropyl alcohol (20.0 g) at, for example, room temperature (i.e. 25° C.). Both the solutions of steps 2 & 3 are combined together and the resulting solution is granulated over the blend of step 1.

4) The granulated blend was dried in Fluid Bed dryer (“FBD”) at an inlet temperature of 50° C. till a loss of drying (“LOD”) value of 3.5-4.0% w/w is obtained.

5) The dried granules are milled (Co-mill) through a, for example, 0.039 inch screen to obtain a uniform sized granules.

6) The screened granules are then blended in a suitable blender with ingredients 11 and 12 which were pre-screened through, for example, a 600 sized screen and further lubricated with ingredient 13 which was pre-screened through, for example, a 425μ screen.

7) The resulting blend is compressed into tablets on a Colton rotary machine using capsule shaped punches.

### Dissolution Testing

Dissolution testing of each of the tablet formulations mentioned hereinabove were done at 37° C. in 900 ml of pH 7.5 phosphate buffer in USP Type II Apparatus at 75 rpm. An example of a dissolution profile is provided hereinbelow.

TABLE 1

| | Time % of Telmisartan Dissolved | | | | |
|---|---|---|---|---|---|
| | | | | | Composition |
| Time | | | | | With dissolving |
| Points | Formula A | Formula B | Formula C | Formula D | matrix |
| 5 min | 25.0 | 30.0 | 31.0 | 26.0 | 30.0 |
| 10 min | 49.0 | 57.0 | 60.0 | 51.0 | 48.0 |
| 15 min | 70.0 | 86.0 | 98.0 | 75.0 | 60.0 |
| 30 min | 92.0 | 96.0 | 98.0 | 94.0 | 89.0 |
| 45 min | 93.0 | 97.0 | 100.0 | 94.0 | 95.0 |
| 60 min | 92.0 | 96.0 | 100.0 | 94.0 | 95.0 |

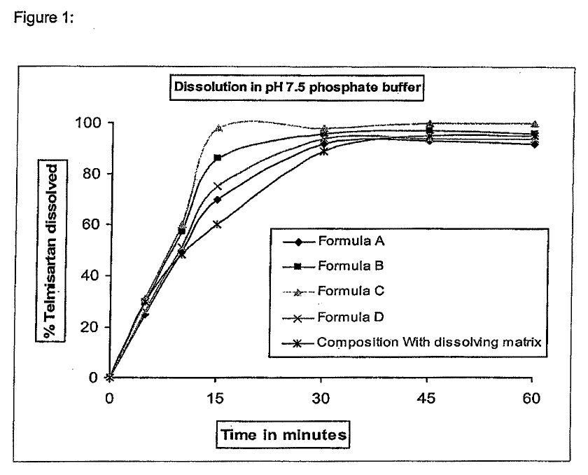

As it can be seen from this data, as well as FIG. 1, Telmisartan is dissolved from the disintegrating matrix; thereby confirming to the quality of the formulation according to the present invention. Moreover, FIG. 1 is a diagram showing the dissolution profile of telmisartan in a phosphate buffer at pH 7.5.

As it may be appreciated from the above and in light of the Examples of the preferred embodiments provided hereinabove, there are many benefits in using a disintegrating matrix as compared to a dissolving matrix. Such advantages include:
(i) a cost effective process as compared to the spray dried or the Fluid bed granulation process; and
(ii) a less hygroscopic and no specialized packaging required to protect the formulation.

The formulation of the present invention may be made by less expensive (conventional) granulation techniques, in comparison to the more expensive techniques of spray drying or fluid bed granulations.

Through physical observation, another advantage of the end-product, i.e. tablet and/capsule, containing the composition and/or formulation of the present invention, is that it is less sensitive to moisture, as opposed to other products (i.e. moisture sensitive products) which need to be packaged in blister packs, and requiring detailed packaging and handling instructions for use of said product. Such an advantage may be attributed in part to the use of a disintegration matrix instead of a dissolving matrix as well as the use of insoluble diluents. As aforementioned, the use of a disintegrating matrix, as opposed to a dissolving matrix, provides many advantages.

While several embodiments of the invention have been described, it will be understood that the present invention is capable of further modifications, and this application is intended to cover any variations, uses or adaptations of the invention, following in general the principles of the invention and including such departures from the present disclosure as to come within knowledge or customary practice in the art to which the invention pertains, and as may be applied to the essential features hereinbefore set forth and falling within the scope of the invention.

1. A stable pharmaceutical composition comprising a pharmaceutically active substance having poor water solubility dispersed in a pharmaceutically acceptable disintegration matrix, said disintegration matrix comprising:
at least one pharmaceutically acceptable disintegrant;
a pharmaceutically acceptable basic agent provided in a molar ratio of basic agent to active substance of 1:1 to 10:1;
a water-insoluble pharmaceutically acceptable diluent;
optionally, if desired or necessary at least one pharmaceutically acceptable excipients and/or pharmaceutically acceptable adjuvants; and
optionally a pharmaceutically acceptable surfactant or emulsifier.

2. The pharmaceutical composition according to claim 1, wherein the active substance having poor water solubility is an angiotensin II receptor antagonist.

3. The pharmaceutical composition according to claim 2, wherein the angiotensin II receptor antagonist is selected from the group consisting of candesartan, eprosartan, irbesartan, losartan, olmesartan, telmisartan, valsartan and mixtures thereof, pharmaceutically acceptable salts thereof or pro-drugs.

4. The pharmaceutical composition according to claim 3, wherein the angiotensin II receptor antagonist is telmisartan or a pharmaceutically acceptable salt thereof.

5. The pharmaceutical composition according to claim 4, wherein the basic agent may be selected from alkali metal hydroxides, NaHCO₃, KHCO₃, Na₂CO₃, 15 Na₂HPO₄, K₂HPO₄, tromethamine, triethanolamine, MgO, MgCO₃ and basic amino acids.

6. A stable pharmaceutical composition comprising 5.0% to 50% by weight of telmisartan dispersed in a disintegration matrix, said disintegration matrix comprising:
at least one disintegrant in an amount ranging from 0.5%-20% by weight;
a basic agent provided in a molar ratio of basic agent to telmisartan of 1:1 to 10:1;
a water-insoluble diluent in an amount ranging from 15%-75% by weight;
at least one member of the group consisting of pharmaceutically acceptable excipients and/or pharmaceutically acceptable adjuvants, in an amount ranging from 0-25% by weight; and
optionally a surfactant or emulsifier present in an amount ranging from 0.5%-10% by weight,
the sum of all components equalling 100.0%.

7. A stable pharmaceutical composition comprising 5.0% to 50% by weight of telmisartan dispersed in a disintegration matrix, said disintegration matrix comprising:
at least one disintegrant in an amount ranging from 2%-20% by weight;
a basic agent provided in a molar ratio of basic agent to telmisartan of 1:1 to 10:1;
a water-insoluble diluent in an amount ranging from 15%-75% by weight;
at least one member of the group consisting of pharmaceutically acceptable excipients and/or pharmaceutically acceptable adjuvants, in an amount ranging from 0-25% by weight; and
optionally a surfactant or emulsifier,
the sum of all components equalling 100.0%.

8. The pharmaceutical composition according to claim 1 further comprising a film coat.

9. The pharmaceutical composition according to claim 8, wherein the film coat comprises at least one film forming polymer selected from the group consisting of hydroxypropylmethylcellulose, ethylcellulose, polyvinyl alcohol, hydroxypropylcellulose, acrylic polymers (Eudragit®), hydroxyethylcellulose, polyvinyl pyrrolidone (Povidone), and vinyl pyrrolidone-vinylacetate copolymer (copovidone), as well as mixtures thereof.

10. The pharmaceutical composition according to claim 9, wherein the water insoluble diluent is selected from the group consisting of microcrystalline cellulose, di- or tri-basic calcium phosphate, meglumine oxide, crystalline cellulose, powdered cellulose, anhydrous silicic acid, calcium carbonate, calcium sulphate, magnesium silicate, magnesium trisilicate, magnesium aluminium metasilicate (Neusilin), kaolin, starch, starch derivatives, magnesium carbonate, magnesium oxide and co-processed insoluble excipients.

11. The pharmaceutical composition according to claim 10, wherein the co-processed insoluble excipient is selected from the group consisting of Ludipress® (mixture of lactose and 3.2% Povidone K30), Cellactose® (mixture of lactose and cellulose), and ProsolV® (mixture of microcrystalline cellulose and silicon dioxide).

12. The pharmaceutical composition according to claim 11, wherein the co-processed insoluble excipient is silicified microcrystalline cellulose.

13. The pharmaceutical composition according to claim 12, wherein the composition comprises an emulsifier selected from the group consisting of MYVACET™ (distilled acetylated mono-glyceride emulsifers); ARLACEL™ (mainly sorbitan esters); TWEEN™ (polyoxyethylene sorbitan esters); CENTROPHASE™ (fluid lecithins); CREMOPHOR™ (polyoxyl castor oil derivatives; or macrogol ethers; or macrogol esters); LABRAFAC™ (caprylic/capric triglyceride); LABRAFIL™ (polyoxyethylated glycolysed glycerides); LABRASOL™ (mixture of mono-, di- and triglycerides and mono- and di-fatty esters of polyethylene glycol. The predominant fatty acids are C8-C10 caprylic/capric acids; MYVEROL™; and TAGAT™ (polyethyleneglycol hydrogenated castor oil; or polyethyleneglycol glyceryl esters); lecithin; and proteins such as casein.

14. The pharmaceutical composition according to claim 12, wherein the composition comprises a surfactant selected from the group consisting of: sodium lauryl sulphate, polyoxyethylene sorbitan fatty acid esters (TWEEN™ series), Myrj™ series, solutol HS, polyoxyethylene alkyl ethers (for example the Brij™ series), polyoxyethylene castor oil derivatives, polyoxyethylene stearates, sorbitan esters (sorbitan fatty acid esters), poloxamers, sucrose fatty acid ester, vitamin E TPGS, and polyethylene glycol fatty acid esters.

15. The pharmaceutical composition according to claim 12, wherein the composition comprises at least one pharmaceutically acceptable excipient and/or adjuvant wherein the at least one pharmaceutically acceptable excipient and/or adjuvant is selected from the group consisting of binders carriers, lubricants, flow agents, adsorbants, crystallization retarders, disintegrants, solubilizers, anti-adherents, surfactants, pH modifiers and coloring agents.

16. Process to prepare a pharmaceutical composition comprising the steps of:
(a) preparing a dry mixture containing at least one water insoluble diluent, an adsorbant, a flow aid, and a disintegrant;
(b) preparing a granulation (or drug-binder) solution, said solution obtained by combining a first mixture containing purified water, at least one basic agent (1:1 to 1:10 molar ratio of basic agent to telmisartan) and a pharmaceutically active substance, to a second mixture containing a binder dissolved in a alcohol;
(c) screening the dry mixture obtained in step (a) through a screen and then charging it into a low shear equipment;
(d) granulating the dry mixture of steps (a) and (c) with granulation solution of step (b) so as to form granules;
(e) drying the so formed granules of step (d) in a fluid bed drier or a tray drier;
(f) co-milling the dried granules of step (e) through a co-mill;
(g) preparing a mixture containing a basic agent, a water insoluble diluent and a disintegrant, all of which will be screened through a sieve;
(h) mixing the mixture of step (g) with the dried granules of step (f) to form a mixture to which a lubricant is added thereon;
(i) compressing said mixture of step (h) into a solid dosage form; and
(j) coating the compressed tablets with Opadry®.

17. The process according to claim 16, wherein step (a) further comprises:
the amount of at least one water insoluble diluent used is in the range of 15%-75% by weight;
the amount of the adsorbant used is in the range of 10%-25% by weight;
the amount of the disintegrant used is in the range of 1.0%-10.0% by weight;
wherein step (b) further comprises:
the amount of the pharmaceutically active substance used is in the range of 5%-50% by weight; and
the amount of binder used is in the range of 15%-10% by weight;
wherein step (g) further comprises:
the amount of the basic agent used is in the range of 1.0%-10% by weight;
the amount of the water insoluble diluent used is in the range of 10%-30% by weight; and
the amount of disintegrant used is in the range of 1.0%-10% by weight;
wherein the amount of the lubricant used in step (h) is in the range of 0.25% to 2.0% by weight; and
wherein the amount of Opadry® used in step (j) is in the range of 1.0%-3.0% by weight.

18. The process according to claim 16, wherein the pharmaceutical composition according to claim 1 is prepared.

19. The process according to claim 16, wherein the pharmaceutical composition according to claim 6 is prepared.

20. The process according to claim 16, wherein the pharmaceutical composition according to claim 7 is prepared.

21. The process according to claim 17, wherein the pharmaceutical composition according to claim 1 is prepared.

22. The process according to claim 17, wherein the pharmaceutical composition according to claim 6 is prepared.

23. The process according to claim 17, wherein the pharmaceutical composition according to claim 7 is prepared.
